(19) United States
(12) Patent Application Publication
(10) Pub. No.: US 2005/0276832 A1
(43) Pub. Date: Dec. 15, 2005

(54) Topical compositions and uses thereof for treating aquatic animals subjected to disinfection and vaccination

(75) Inventors: Luis Arrieta Castroviejo, Santiago (CL); Samuel Valdebenito Contreras, Santiago (CL)

Correspondence Address: DARBY & DARBY P.C., P. O. BOX 5257, NEW YORK, NY, 10150-5257, US

(73) Assignee: Veterquimica Ltda., Santiago (CL)

(21) Appl. No.: 11/150,438

(22) Filed: Jun. 9, 2005

(30) Foreign Application Priority Data
Jun. 9, 2004 (CL) 1432-2004

(51) Int. Cl. A61K 31/19; A61K 31/14; A01N 31/00

(52) U.S. Cl. 424/401; 514/643; 514/738; 514/557

(57) ABSTRACT

The present invention is related to compositions that are useful for optimizing the anti-microbial activity of solutions of disinfectants and biocides, and promoting the absorption of antigens from immersion vaccinations for fishes. The compositions comprise a compound with a moisturizing activity, a compound with exfoliating activity, and at least one biocide (e.g., an agent that acts against bacteria, viruses, fungi, parasites, and/or combinations thereof).

### FIELD OF THE INVENTION

This invention is related to compositions that are useful for optimizing the anti-microbial activity of solutions of disinfectants and biocides, and promoting the absorption of antigens from immersion vaccinations for fishes. The compositions comprise a compound with a moisturizing activity, a compound with exfoliating activity, and at least one biocide (e.g., an agent that acts against bacteria, viruses, fungi, parasites, and/or combinations thereof).

### BACKGROUND OF THE INVENTION

The industrial cultivation of fish is an activity that is expanding dynamically, especially in relation to the salmon species. Two of the most acute sanitary problems affecting the producers are:
(1) The high rate of infections with microorganisms and parasites that affect the corporeal mass of the fish, especially in the primary stages of the industrial cultivation processes in the fresh water stage. Among these types of microorganisms, the strongest incidence corresponds to a parasitic fungi of the Saprolengia genre producing fungal epidemic diseases in the fish. This favors attacks by other pathogenic microbial species, among which the most outstanding are viruses (e.g., IPNv), rickettsias (e.g., pisciricketssia salmonis), and bacteria (e.g., yersinia ruckeri, streptococcus, and flavobacterium).
(2) The infectious diseases produced by pathogens constitute the second major problem that the industrial aquatic establishments must overcome.

A very common procedure to preserve the sanitary condition of the fish in the cultivation pens consists of sanitizing the fish every so often with immersions in diluted solutions of disinfectants. Until it was banned recently, the most commonly used agent was malachite green, a compound that shows excellent antifungal properties, but whose use was forbidden because of its risks for human health due to its possible teratogenic and/or carcinogenic activity. This caused an important gap in the selection of available disinfectants that could be applied routinely at the salmon industrial plants, stimulating the research and development of new disinfectants.

On the other hand, the infectious diseases that affect the salmonoid fish and produce the most important losses are: Infectious Pancreatic Necrosis produced by the virus (IPNv), and the diseases produced by bacterial pathogens belonging mainly to the genre Yersinia, Streptococcus, Flavobacterium, Ricketssias, and Aeromonas. These pathogens, which affect a large variety of fish and other aquatic organisms, show a cosmopolitan distribution and have been activated extraordinarily as a result of the industrial massification of salmon breeding developed in the last twenty years. This produces serious losses due to a drastic drop in the yields of final harvest at the farms. (Laider, L. A. 2001, Microbiological Problems in the Salmon Processing Industry, in: Kestin, S. C. and Warris, P. D. (eds.) “Farmed Fish Quality,” Blackwell Sci. Inc. (USA)).

To prevent the appearance of these infectious diseases, the obvious and recurrent strategy is the application of vaccines, based on the immunology principles applied in human and animal health since the pioneering discoveries of Pasteur in the XIX Century. Practically 100% of the vaccines currently in use in aquiculture correspond to those known as BACTERINES or VIRINES, e.g., concentrated and inactivated cultures of the same pathogen organism that produces the disease. The recombinant vaccines, obtained from the isolated DNA of pathogen strains, is a biotechnique that is increasingly growing at the research laboratory level, but whose field application is still under heavy discussion. In the case of the strategies applied to the field of industrial aquiculture, the vaccines are the actual motor into the fight to prevent infectious diseases. Currently the industrial plants apply highly developed vaccination programs under the careful and regulated control of the sanitation authorities.

There are two ways in which fish can be vaccinated: the vaccines can be applied by injection or by immersion. At present, one of the biggest problems in aquiculture is that the injectable vaccines cannot be used in fish weighing less than 15-20 grams due both to the risk of producing intra-peritoneal adhesions and to the production of high dispersion in fish sizes at harvesting. This means that fishes between 4 grams (age at which the immune system of the individual is already mature) and 20 grams remain fully exposed to the opportunistic pathogens, especially at the stage when they are growing in fresh water.

In the scheme included as FIG. 1, a summary of this situation is illustrated for the specific case of the IPN virus. This schematic presentation indicates that if the IPN virus affects the fish within this period—which is approximately 5 to 8 weeks precisely when the presence of IPN is highly frequent—the sanitary damage becomes practically irreversible because subsequent vaccinations are poorly efficient since the fish are immunosupressed as the consequence of IPN. In salmon producing countries located in the northern hemisphere, the IPN virus is mainly active at the saltwater phase; therefore the vaccines developed are injectable for fish larger that 15-20 grams. Nevertheless, in countries in the southern hemisphere (e.g., Chile), this disease, like others, appears during the freshwater stage. Therefore, there is an urgent need to develop vaccines that are applied by immersion to broaden the period of vaccination to younger fish weighing less than 15 grams. In this way, the producer will have a vaccinal tool to protect his sanitary patrimony from the moment in which the fish has a mature immune system, preventing infection exactly in the periods of maximum risk of infection.

In spite of the clear advantages of the immersion vaccines, from an epidemiological and operative point of view, it has been the injectable vaccines that have had a greater development in salmon breeding. The intraperitoneal injection of fish is currently the most common method to administer vaccines, principally due to its high efficiency and meticulous dosing that guarantees adequate plasmatic levels. The current immersion vaccines are characterized by their inefficient absorption through the fish's skin, which is the physiologically most active organ to incorporate the antigen formulated as an immersion vaccine. The disadvantages of the injectable vaccines arise especially from the generation of stress in the fish, the high cost of labor, the time required for their application and the risks of accidents that may affect both the fish that are being injected as well as the operators. Nevertheless, the main disadvantage of injectable vaccines is the impossibility to apply them to young specimens, as stated previously.

On the contrary, the immersion vaccines are highly recommendable for mass vaccination of small fishes, even when the immunoprotection must be achieved with higher doses of antigens due to their low levels of absorption. It has been proven that the principal absorption route of antigens from the immersion vaccines is the skin, while the absorption through the fish's gills is secondary (Moore et al., Particulate antigen uptake during immersion immunization of fish: the effectiveness of prolonged exposure and the roles of skin and gill. Fish Shellfish Immunology. 1998; 8:393-407; Smith, P. A. Experimental infection of coho salmon by exposure of skin, gills and intestine with Piscirickettsia salmonis. Dis. Aq. Organisms. 2004, 61:53-57).

As a consequence of the important advantages of immersion vaccines for fish, their development has been recently highly stimulated (Nakanishi T., Ototake M. Antigen uptake and immune responses after immersion vaccination. In: Gidding R., Lillehaug A. et al., eds. Fish Vaccinology, Basel: Karger, 1997, 59-68; Ototake et al., Prolonged immersion improves the effectiveness of dilute vibrio vaccine for rainbow trout. Kiryu et al., Fish Pathol. 1999: 34, 151-154. The uptake of fluorescent microspheres into the skin, fins and gills of rainbow trout during immersion. Fish Pathol. 2000: 35, 41-48). Therefore, in developing modern vaccine programs (both bacterine as well as recombining), the first option is the choice of a pharmaceutical form whose antigen can be applied via immersion (Gidding et al., Recent developments in fish vaccinology. Vet Immunol Immunopathology 1999; 72, 203-212; IPEVAC-Inmersión, Exp. Veterquímica, 2004), except for the vaccines that must be applied to fish of high weights.

Numerous efforts have been developed to increase the absorption efficiency of immersion vaccines for fish, taking into consideration the progress achieved earlier in the percutaneous application of vaccines in humans (Rosenthal, S. R., The multiple puncture method of BCG vaccination. Am. Rev Tuberculosis, 1939: 39, 128-134). These efforts have been oriented mainly toward increasing the absorption capacity of the skin by the production of multiple acupunctures, thereby causing a clear increment in the antigen absorption through these superficial dermal lesions, taking care not to cause very deep perforations. An instrument for multiple acupuncturing of fish was described recently (Nakanishi et al., Development of a new vaccine delivery method for fish: percutaneous administration by immersion with application of a multiple puncture instrument, Vaccine 20, 2002: 3764-3769) and is applied prior to the bath with the immersion vaccine. The authors report a substantial increase in the efficiency of this procedure, in spite of its bloody nature which goes against the modern postulate that does not accept the mistreatment of animals.

In brief, the availability in the market of efficient immersion vaccines is highly needed to improve the control of the infectious diseases that affect aquiculture.

The system and means of the present invention refer to the development of innovative compositions that contain disinfectants or biocides (preferably formalin and quaternary derivatives of zalconium) and other water-soluble organic agents that have moisturizing and exfoliating effects on the skin of the fish (preferably glycerin and glycolic acid, respectively). Other suitable biocides include, but are not limited to, compounds selected from aldehydes and compounds derived from quaternary ammonium or halogenated organic compounds (preferably benzalconium chloride).

The topical application of the present compositions synergistically enhances the effect of disinfectant agents at very low doses. These novel compositions are applied advantageously (as compared to other compositions known in the art which do not include combinations of moisturizing and exfoliating agents), obtaining excellent results at low doses of the disinfecting agents to preserve the sanitary quality of farmed fish. The disinfectant property of these novel compositions is exemplified in dealing with a Saprolegnia parasiti fungus, a pathogen that currently affects severely the salmonidae in their industrial breeding pens.

These novel compositions can be used simultaneously as an antigen carrier-like vehicle applicable to fish by immersion to prevent infectious diseases, making it easier to apply a procedure that permits a sharp increase in the efficiency of immersion vaccines for fish, favoring the absorption of antigens through the skin and gills, organs that are pre-conditioned by the synergic action of the disinfectant, humectant and exfoliating agents. The results obtained from the present compositions support the claim that with these compositions the efficiency of the immersion vaccines improves notably, broadening their range of protection. These results are exemplified with the immersion vaccines against Flavobacterium psychrophyllum and against IPNv.

The following U.S. patents are related to the use of different disinfectants or mixtures thereof for use in veterinary or human medicine:

U.S. Pat. No. 6,160,023 relates to compositions that include bronopol (bromo-2-nitropropane-1.3-diol) to treat several diseases of aquatic organisms, including among them the parasitic Saprolengia).

U.S. Pat. Nos. 6,117,457 and 6,365,170 relates to the use of disinfectant solutions of peracetic acid and hydrogen peroxide.

U.S. Pat. No. 6,414,023 relates to disinfectant compositions that include 2,4-dichlorobenzyl alcohol and glycerilmonolaurate in a hydroalcoholic vehicle.

U.S. Pat. No. 6,518,252 relates to an immersion and bath method to reduce the infection load of an aquatic animal applying an antibiotic solution with antibacterial properties increased with the inclusion in the composition of at least one chelating agent and optionally a buffer agent. The chelating agent is disclosed as having three functions:
a) reduction of the levels of divalent metallic ions in the water that may inactivate the antibiotic;
b) removal of metallic ions from the cellular walls of the microorganism, thus weakening the resistance of that microorganism to the penetration of anti-microbial agents; and
c) creation of pores in the cellular wall which, while making it easier for the antibiotic to enter, also favors the exit of the microbic intracellular components, all of which reduces the viability of the hostile microorganisms.

U.S. Pat. No. 6,893,667 relates to a method to deliver a preparation of powdered nucleic acid molecules into vertebrate tissue for transformation of cells in the tissue using needleless injection techniques.

U.S. Pat. No. 6,887,989 relates to a fish vaccine to protect salmon against infection by Piscirickettsia salmonis based on the nucleic acid or amino acid sequence of antigens from the same microbe.

U.S. Pat. No. 6,872,386 relates to an oral vaccine that includes a multiple-cell organism for use as food for an aquatic animal to be vaccinated, and a single-cell organism fed to, and as a result, bioencapsulated by, the multiple-cell organism.

U.S. Pat. No. 6,855,372 relates to a method and apparatus for coating skin piercing microprojections.

U.S. Pat. No. 6,673,374 relates to an anti-microbial composition for the treatment of human skin affected by dermatitis of different etiologies, made from hydrogen peroxide, one or more moisturizing agents and an anti-inflammatory agent; and optionally including an exfoliating agent.

U.S. Pat. No. 6,699,907 relates to a composition of anti-microbial action made from a polar lipophilic solvent and fatty acids with 8 to 14 carbons.

U.S. Pat. No. 4,009,259 relates to a method for treating fish and increasing the efficiency of vaccines or disinfectants. The fish are submerged in hyperosmotic solutions for 2 to 3 minutes and are then submerged in another solution of the immersion vaccine or chemotherapeutic solutions. These solutions are made from sodium, potassium, calcium and magnesium salts under the form of sulfates, chlorides or phosphates.

U.S. Pat. No. 4,223,014 relates to a method to immunize fish by applying spray vaccines directly on the fish; it includes the application, using this system, of inactivated vaccines of strains of Vibrio anguillarum, Aeromonas salmonicida and furunculosis.

U.S. Pat. No. 4,282,828 relates to an apparatus to apply immersion vaccines in fish by the direct aspersion of the vaccine in a spray. The fish are confined in a rectangular receptacle where they are exposed to such a bathing procedure.

U.S. Pat. No. 4,287,179 relates to a procedure to apply an immersion vaccine for the red mouth disease (yersiniosis) by simply applying the vaccine in the form of a non-pressurized immersion.

U.S. Pat. No. 4,363,290 relates to an automatic apparatus to apply one or more immersion vaccines, which consists of a means to lead the fish into a compartment containing the vaccinal solution and later, once the required immersion contact time is completed, divert the fish toward their outside habitat.

U.S. Pat. No. 5,498,414 relates to the administration of vaccines against the furunculosis of fish by immersion in solutions of attenuated strains of Aeromonas salmonicida.

### BRIEF DESCRIPTION OF THE FIGURES

FIG. 1 is a summary scheme of the active cycles of IPNv infection in salmon.

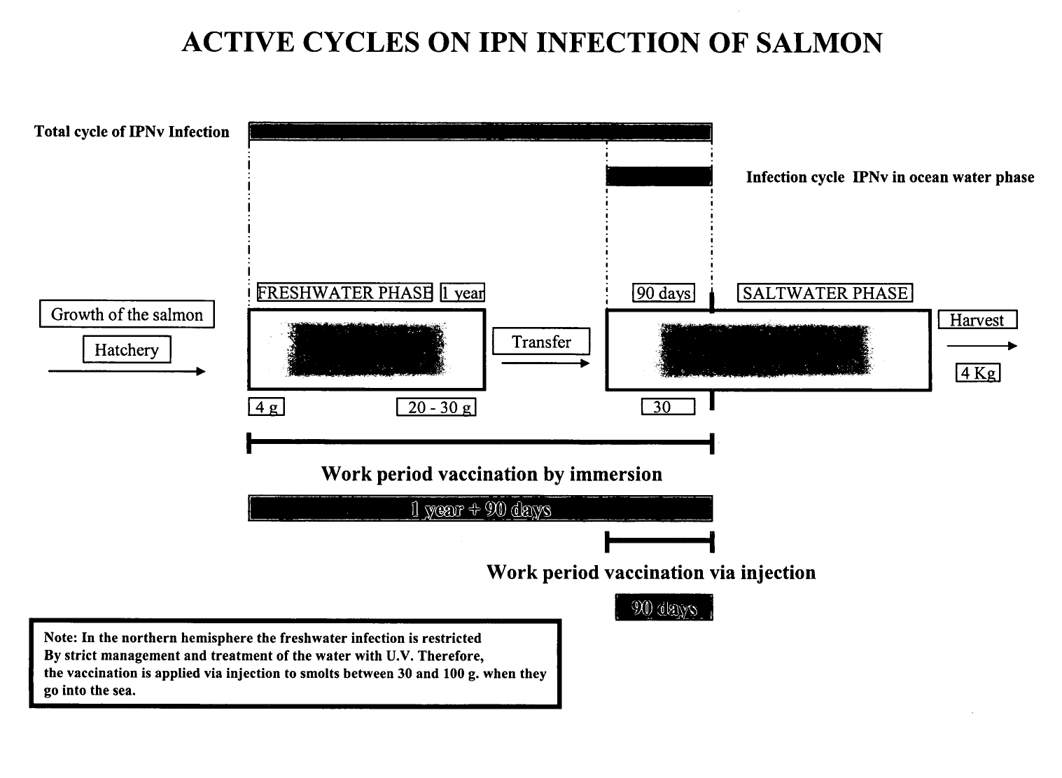

### DETAILED DESCRIPTION OF THE INVENTION

One objective of the present invention is to provide a composition to optimize both the anti-microbial activity of disinfectant solutions, enhancing their efficacy at low doses, and/or the efficacy of immersion vaccines for farmed fish. Optimizing anti-microbial activity and/or efficacy of immersion vaccines includes, but is not limited to, maintaining and/or increasing these conditions.

Another objective of this invention comprises providing a composition that includes a moisturizing agent (preferably glycerin), an exfoliating agent (preferably glycolic acid), and one or more anti-microbial or anti-parasitic agents (preferably formalin and benzalconium chloride) to improve both the sanitary quality of farmed fish (preferably directed toward fighting infections produced by the genre Saprolengia on the skin and gills of fish), and/or to improve the absorption of immunogens contained in immersion vaccines.

Another objective of this invention is to provide a composition to prevent or reduce the occurrence of infections produced by other pathogen agents developed inside fish.

The principal advantage of the composition of this invention comprises optimizing the germicide and/or anti-parasitic effects of biocide compounds, thus broadening the range from which such agents may be selected by reducing considerably the doses required to achieve significant effects with the biocides and/or improving the efficacy of immersion vaccines for fishes. This also favors environmental conservation because the levels of chemicals eliminated into watercourses or by other means are minimized and easily biodegraded.

Alcohols with a larger number of OH functional groups constitute a group of organic molecules that are distinguished in dermatology and cosmetics because of their excellent skin softening and moisturizing properties. Among these, the most outstanding are glycerin and the glycols, especially the propylene glycols. Glycerin is a tri-alcohol (propanetriol) soluble in water and widely used in pharmaceutical preparations as a solvent and humectant. On their part, the organic acids are distinctive as they are also used in dermatology and cosmetics because of their skin exfoliating properties, due to their capacity to remove the first layer of old epithelial cells. Within this group of chemical products, the most outstanding is glycolic acid. It corresponds to hydroxyacetic acid with the formula HOCH₂COOH, and has the property of irritating the skin very gently and is used a great deal in human cosmetology as an exfoliating agent. Other suitable exfoliating compounds include, but are not limited to, lactic acid, citric acid, salicylic acid, and tannic acid.

The total amount of disinfectant(s) and/or biocide(s) used in the present invention is less than or equal to about 425 ml per about 1000 ml±about 500 ml of distilled water c.s.p. In one embodiment, the total amount of disinfectant(s) and/or biocide(s) is about 200 ml±about 100 ml. In another embodiment, the total amount of disinfectant(s) and/or biocide(s) is about 208±about 104 ml. In another embodiment, the total amount of disinfectant(s) and/or biocide(s) is about 8 ml±about 4 ml, preferably about 8 ml. In another embodiment, the total amount of disinfectant(s) and/or biocide(s) is about 16±about 8 ml, preferably about 16 ml. One preferred disinfectant, formalin, may be used in an amount of about 200 ml±about 100 ml, preferably about 200 ml. Another preferred disinfectant, benzalconium chloride, may be used in an amount of about 4 ml to about 24 ml, preferably about 8 ml to about 16 ml.

The amount of moisturizing agent used in the present invention can be about 10 ml to about 60 ml per about 1000 ml±about 500 ml of distilled water c.s.p. In one embodiment, the amount of moisturizing agent is about 20 ml±about 10 ml, preferably about 20 ml. In another embodiment, the amount of moisturizing agent is about 40 ml±about 20 ml, preferably about 40 ml.

The amount of exfoliating agent used in the present invention can be about 100 ml to about 600 ml per about 1000 m±about 500 ml of distilled water c.s.p. In one embodiment, the amount of exfoliating agent is about 200 ml±about 100 ml, preferably about 200 ml. In another embodiment, the amount of exfoliating agent is about 400 ml±about 200 ml, preferably about 400 ml.

The current invention is a composition that combines both types of compounds in such a proportion that the moisturizing synergic action, obtained preferably with glycerin, favors the smooth action on the skin of the exfoliating agent, preferably glycolic acid, which is present in low concentrations. The result of this novel combination in the field of disinfectant solutions provides a global emollient action on the skin. In this way, the skin of the entire fish is much more susceptible to the action of disinfectants, efficiently destroying the microorganisms adhering to the surface or the more internal layers of the skin of the animals treated. In this way, the disinfectant agents can be applied in lower doses, with a broad microbicide activity and without adverse collateral effects.

In this invention, compositions of formalin and benzalconium chloride are used preferably for the treatment of fish, which does not constitute limiting the invention to only those disinfectants and animals. All those knowledgeable in the art will understand that this innovating system and means—based on the synergetic moisturizer-exfoliating effect, represented especially by the synergic composition of glycerin and glycolic acid—can be extended to other disinfecting agents and to other species of animals.

Concurrently with the disinfectant and preventive action of the novel compositions of the present invention, it has been proven experimentally that the EXFOLIATIVE-HUMECTANT-TENSOACTIVE (EHT) synergic action on the skin of the fish favors the absorption of the antigen or antigens of the immersion vaccine with which the fish is being treated. In this way, the skin of the entire fish remains more suitable for absorbing the antigens that are the components of the formulation of a vaccine that is applied by immersion, causing a significant improvement in the efficiency of said vaccine.

The system and means of this invention is applied to breeding fish in captivity, especially the industrial breeding of salmon and trout. In the plants where these fish are bred, the contamination with microbial agents is a serious limitation to production because it results in important losses due to morbidity and/or mortality of the fish population. As is proven, explained and illustrated in more detail with examples herein, the compositions of disinfectants (formulated preferably on the basis of formalin, benzalconium chloride, glycerin and glycolic acid) present a high anti-microbial activity in low doses, without producing ecological damage. The tests with fish reported in this patent have been carried out experimentally at a marine development plant and in the experimentation aquariums of the Veterquímica Laboratory. The different treatments were tested using fish of the Atlantic salmon species, obtaining excellent results with regard to the disappearance of infections caused by Saprolengia spp. The anti-fungi action of these preparations was 200% more effective than the current leading product in this field and whose active principle is bronopol.

The immersion vaccination techniques used in accordance with the present invention are those that are conventionally utilized by those of skill in the art. The immersion time may vary from about 30 seconds to about 2 hours. The present compositions may be applied before the immersion vaccine is applied by bathing the fish in present composition for about 5 minutes to about 90 minutes, preferably for about 15 minutes to about 30 minutes.

The present invention is next described by means of the following examples. The use of these and other examples anywhere in the specification is illustrative only, and in no way limits the scope and meaning of the invention or of any exemplified form. Likewise, the invention is not limited to any particular preferred embodiments described herein. Indeed, modifications and variations of the invention may be apparent to those skilled in the art upon reading this specification, and can be made without departing from its spirit and scope. The invention is therefore to be limited only by the terms of the claims, along with the full scope of equivalents to which the claims are entitled.

Specifically, the following examples of immersion vaccines against the virus IPN and the bacteria Flavobacterium psychrophillum does not mean that the invention is limited to only these pathogen vaccines. All those well versed in the art will understand that this novel treatment and means is based on a synergic effect (humectant-exfoliant-tensoactive, preferably represented by the synergic composition of glycerin, glycolic acid and benzalconium chloride) and can be extended to other vaccines and against other pathogens.

### EXAMPLES

Formulations Used as Disinfectants

Three formulations were prepared as follows:

Formulation A:

Formalin, 40% solution 200 ml
Benzalconium chloride, 50% solution 8 ml
Glycerin 20 ml
Glycolic acid 200 ml
Distilled water c.s.p. 1000 ml
   Formulation B:
Formalin, 40% solution 200 ml
Glycerin 20 ml
Glycolic acid 200 ml
Distilled water c.s.p. 1000 ml
   Formulation C:
Formalin, 40% solution 200 ml
Benzalconium chloride, 50 solution 8 ml
Glycerin 20 ml
Distilled water c.s.p. 1000 ml

Formulations A, B, and C were subjected to the following experimental tests:

### Example 1

Five groups were formed, made up of 30 fish each of the Atlantic salmon species, 40-50 grams, showing clear signs of infection by the fungus Saprolengia sp., confirmed by laboratory tests. One of the groups was preserved as control without treatment, while the four remaining groups were subjected to experimental antifingal treatments. In this way, the groups were set up as follows:
GROUP 1: Without antifungal treatment
GROUP 2: Treated with the antifungal product bronopol, according to recommended dose of 20 ppm
GROUP 3: Treated with the Formulation A, in a dose of 28 ml/70 liters of water
GROUP 4: Treated with the Formulation B, in a dose of 28 ml/70 liters of water
GROUP 5: Treated with the Formulation C, in a dose of 28 ml/70 liters of water

The treatments consisted of the following: on Day 1, the fish were submerged for one hour in a bath with 70 liters of water to which the corresponding formulation had been added. Afterwards, they were transferred to breeding tanks that only contained water and with a standard feeding regime, all under the customary aquarium control that includes controlling the temperature (16-17 degrees C.), dissolved oxygen levels not lower than 7 ppm, and control of nitrites and ammonium. On Day 7, the fish were subjected to a second bath, similar to that of Day 1, to continue their development until 14 days of breeding had been completed, keeping a record of the fish killed due to the effect of the Saprolengia fungus. On Day 15, the fish that survived were harvested and the level of infection by Saprolengia was determined for each fish.

The infection level was classified according to the following scale:
INFECTION GRADE 0: healthy fish, without fungus on the skin
INFECTION GRADE 1: fish having only fungal spots on the skin.
INFECTION GRADE 2: fish with short fungal hypha
INFECTION GRADE 3: fish having necrosis of the tissues
INFECTION GRADE 4: fish with cottony hypha
INFECTION GRADE 5: fish dead due to fungal infection (occurring during the 14 days of breeding)
   Results

The condition of the fish that survived, after the 14 days of breeding, was classified according to the previous scale, and the following results were obtained:

| | | |
|---|---|---|
| GROUP 1 (without treatment) | fish with grade 5 (death) | 70% |
| | fish with grade 1-2 | 30% |
| GROUP 2 (bromopol) | fish with grade 5 (death) | 50% |
| | fish with grade 1-2 | 40% |
| | fish with grade 0 (healthy) | 10% |
| GROUP 3 (Formulation A) | fish with grade 5 (death) | 10% |
| | fish with grade 1 | 30% |
| | fish with grade 0 (healthy) | 60% |
| GROUP 4 (Formulation B) | fish with grade 5 (death) | 25% |
| | fish with grade 1-2 | 40% |
| | fish with grade 0 (healthy) | 35% |
| GROUP 5 (Formulation C) | fish with grade 5 (death) | 40% |
| | fish with grade 1-2 | 48% |
| | fish with grade 0 (healthy) | 12% |

### Example 2

The same above-mentioned “in-vivo” tests with fish were repeated using Formulation A, but using bromopol instead of the two antifungal agents (formalin and benzalconium chloride). With this biocide, the result was identical to that reported above for Formulation B, which proves that the combination (humectant-exfolient) effectively constitutes a system and highly efficient means to optimize the action of disinfectant compounds.

Conclusions

Formulation A, made up of the three active agents (i.e., a dual antifungal (formalin and benzalconium chloride), an exfoliating agent (glycolic acid), and a skin moisturizing agent (glycerin)) presented the best efficiency to counteract the fungal infection. The bath with this formulation after 14 days significantly reduced fish death due to Saprolengia sp., from 70% to 10%.

### Example 3

The same previous examples were repeated with formulations wherein other moisturizing agents were used, such as sorbitol and propylene glycol, other exfoliating agents, such as citric acid, salicylic acid and tannic acid, and other disinfectant chemicals, such as hydrogen peroxide and bronopol. The results obtained were similar to those of Examples 1 and 2, but at higher doses.

Formulations Used as Adjuvants of Immersion Vaccines

Two formulations were prepared as follows:

Formulation A: Solution EHT-A

Glycolic acid 200 ml
Glycerin 20 ml
Benzalconium chloride, 50% sol. 8 ml
Distilled water c.s.p. 1000 ml
   Formulation B: Solution EHT-B
Glycolic acid 400 ml
Glycerin 40 ml
Benzalconium chloride, 50% sol. 16 ml
Distilled water c.s.p. 1000 ml

These formulations were used in the following experimental tests:

### Example 4

Eight 70-liter vaccinal baths were prepared (two series with 4 tanks each) to treat 50 fish in each one. Their composition is shown in Tables 2 and 3 below. The 50 fish were immersed in the respective tanks for two minutes, and at the end of that time they were returned to the habitual growing tanks (with a capacity of 70 liters). After 21 days of growth, one series was challenged with the pathogen Flavobacterium psychcrophilum and the other with the IPN virus by means of an intra-peritoneal injection of a dose equivalent to a Lethal Dose 50, conforming strictly to the technical standards established for potency tests. (Gudding R. et al., 1996, Fish Vaccinology. Developments in Biological Standardization. Vol. 90:179-188). After 28 days, a record was made of the number of fish dead due to the effect of the corresponding pathogen. Table 4 shows the results of these tests.

### Example 5

The same vaccination and challenge tests conducted in Example 4 were adapted to the prior application of the EHT combinations indicated in Table 1 (in absence of a vaccine) bathing the fish for two minutes in said solutions. After this, the fish were subjected to vaccination by immersion without EHT and to the corresponding subsequent challenge.

TABLE 1
EHT Compositions for Pre-Treatment of
Fish, Before Vaccinating by Immersion

| | Composition | Composition | Water (c.s.p.) |
|---|---|---|---|
| Formulation | EHT-A (ml) | EHT-B (ml) | (L) |
| A | 28 | 0 | 70 |
| B | 0 | 28 | 70 |

TABLE 2
Composition of Mixed Vaccinal Baths (Vaccine + EHT)
Against Flavobacterium psychrophillum

| | Immersion | | | |
|---|---|---|---|---|
| | Vaccine | EHT-A Sol. | EHT-B Sol. | Water |
| | (L) | (ml) | (ml) | (L) |
| Bath 1: Control | 0 | 0 | 0 | 0 |
| without vaccines | | | | |
| Bath 2: Control | 7 | 0 | 0 | 63 |
| normal vaccine | | | | |
| Bath 3: | 7 | 28 | 0 | 69.97 |
| Experiment 1 | | | | |
| Bath 4: | 7 | 0 | 28 | 69.97 |
| Experiment 2 | | | | |

TABLE 3
Composition of Mixed Vaccinal Baths
(Vaccine + EHT) Against IPNv Virus

| | Immersion | | | |
|---|---|---|---|---|
| | Vaccine | EHT-A Sol. | EHT-B Sol. | Water |
| | (L) | (ml) | (ml) | (L) |
| Bath 5: Control | 0 | 0 | 0 | 70 |
| without vaccines | | | | |
| Bath 6: Control | 7 | 0 | 0 | 63 |
| normal vaccine | | | | |
| Bath 7: | 7 | 28 | 0 | 69.97 |
| Experiment 1 | | | | |
| Bath 8: | 7 | 0 | 28 | 69.97 |
| Experiment 2 | | | | |

TABLE 4
Results Post-Challenge Vaccination
PERCENTAGE OF FISH DEAD 28 DAYS AFTER CHALLENGE
Immersion Vaccine Flavobacterium psychrophilum

| | | |
|---|---|---|
| Bath 1 | Control without vaccines | 76.4% |
| Bath 2 | Control normal vaccine | 32.1% |
| Bath 3 | Vaccine with EHT-A | 19.8% |
| Bath 4 | Vaccine with EHT-B | 20.1% |

Immersion Vaccine IPNv

| | | |
|---|---|---|
| Bath 5 | Control without vaccines | 56.4% |
| Bath 6 | Control normal vaccine | 15.0% |
| Bath 7 | Vaccine with EHT-A | 7.8% |
| Bath 8 | Vaccine with EHT-B | 7.7% |

The results did not show a significant statistical difference between Examples 4 and 5. Table 3 reports the averages of the results of Examples 4 and 5.

Conclusions

The immersion vaccines in combination with both Formulation A and Formulation B were more efficient in the prevention of the effect of the pathogens in respect of the immersion vaccines applied without these formulations. The baths with these formulations, both prior to the vaccination as well as in a mix with the vaccine, after 28 days caused an appreciable improvement in the efficacy of the respective immersion vaccines, both against the bacteria Flavobacterium as well as against the IPN virus.

All references cited and/or discussed in this specification are incorporated herein by reference in their entirety and to the same extent as if each reference was individually incorporated by reference. To the extent that a conflict may exist between the specification and a cited reference, the language of the disclosure made herein controls.

1. A composition comprising a compound with moisturizing activity, a compound with exfoliating activity, and one or more biocides that act against bacteria, viruses, fungi, parasites, and/or combinations thereof;
wherein said composition promotes the absorption of antigens from an immersion vaccine into fish in confinement.

2. The composition of claim 1, wherein the moisturizing compound is selected from alcohols having more than one OH functional group.

3. The composition of claim 1, wherein the moisturizing compound is glycerin.

4. The composition of claim 1, wherein the compound with exfoliating activity is selected from glycolic acid, lactic acid, citric acid, salicylic acid, and tannic acid.

5. The composition of claim 4, wherein the compound with exfoliating activity is glycolic acid.

6. The composition of claim 1, wherein the biocide comprises compounds selected from aldehydes and compounds derived from quaternary ammonium or halogenated organic compounds.

7. The composition of claim 6, wherein the compound derived from quaternary ammonium is benzalconium chloride.

8. The composition of claim 1, wherein the biocide is formalin.

9. The composition of claim 1, wherein the total amount of biocide in the composition is less than or equal to about 425 ml per about 1000 ml±about 500 ml of distilled water.

10. The composition of claim 7, wherein the amount of benzalconium chloride is about 8 ml to about 16 ml per about 1000 ml±about 500 ml of distilled water.

11. The composition of claim 8, wherein the amount of formalin is about 200 ml±about 100 ml per about 1000 ml±about 500 ml of distilled water.

12. The composition of claim 1, wherein the amount of moisturizing agent is about 20 ml±about 10 ml per about 1000 ml±about 500 ml of distilled water.

13. The composition of claim 1, wherein the amount of exfoliating agent is about 200 ml±about 100 ml per about 1000 ml±about 500 ml of distilled water.

14. A method of optimizing the anti-microbial activity of a disinfectant and/or a biocide comprising introducing the composition of claim 1 into a container of fish in confinement in an amount effective to preserve the sanitary quality of the fish.

15. A method of optimizing the anti-microbial activity of a disinfectant and/or a biocide and promoting the absorption of antigens from an immersion vaccine into fish in confinement comprising bathing the fish with the composition of claim 1 before applying the immersion vaccine.

16. The method of claim 15, wherein the immersion vaccine is selected from flavobacterium psychrophilum vaccine and IPNv vaccine.

17. The method of claim 15, wherein the fish are bathed for about 5 minutes to about 90 minutes.

18. A method of optimizing the anti-microbial activity of a disinfectant and/or a biocide and promoting the absorption of antigens from an immersion vaccine into fish in confinement comprising incorporating the composition of claim 1 into an immersion vaccine and then vaccinating the fish by immersion.

19. A method of preventing or reducing the occurrence of infections developed inside fish, comprising introducing an effective amount of the composition of claim 1 into a container of fish in confinement.

20. The method of claim 19, wherein the infection is caused by the fungus Saprolengia sp.
